(19) United States
(12) Patent Application Publication
(10) Pub. No.: US 2006/0039895 A1
(43) Pub. Date: Feb. 23, 2006

(54) Ex-vivo rescue of hematopoietic stem cells after lethal irradiation

(75) Inventor: John P. Chute, Chapel Hill, NC (US)

Correspondence Address: LARGE SCALE BIOLOGY CORPORATION, 3333 VACA VALLEY PARKWAY, SUITE 1000, VACAVILLE, CA, 95688, US

(73) Assignee: LARGE SCALE BIOLOGY CORPORATION, Vacaville, CA (US)

(21) Appl. No.: 11/047,265

(22) Filed: Jan. 31, 2005

Related U.S. Application Data
(60) Provisional application No. 60/548,247, filed on Feb. 28, 2004.

(51) Int. Cl. C12N 5/08; A61K 35/14

(52) U.S. Cl. 424/93.7; 435/372

(57) ABSTRACT

A method of autologous rescue of an individual exposed to a harmful hematopoietic stem cell condition is described. The method includes removing hematopoietic stem cells from the individual, culturing the hematopoietic stem cells and reintroducing cultured hematopoietic stem cells into the individual. Stem cells are grown in a stromal-cell free medium. The harmful condition can be radiation. The culture medium can contain any of several growth stimulating factors.

### PRIORITY CLAIM

This application is a continuation of provisional patent application Ser. No. 60/548,247, filed Feb. 28, 2004, which is hereby incorporated herein by reference in its entirety.

### BACKGROUND OF THE INVENTION

1. Field of Invention

The present invention relates the culturing of treated bone marrow cells and their use autologously.

2. Description of Prior Art

The potential for purposeful misuse of ionizing radiation as a terror weapon has increased public awareness of the biological effects of radiation injury [1-4]. Despite the renewed focus on the potentially lethal myeloablative effects of high dose ionizing radiation exposure [1,3,4], little has been published addressing treatment options for exposed individuals. As detailed following the recent criticality nuclear accident in Japan [5], supportive therapy with antibiotics and blood products is often inadequate for patients exposed to doses above 600 cGy and allogeneic stem cell transplantation remains the only definitive treatment option [1,5]. However, in mass casualty scenarios, HLA-matched allogeneic stem cell donors would not be available for many victims and the practical administration of allogeneic stem cell transplants in a timely manner would be logistically difficult.

Experimental models have delineated the toxic effects of ionizing radiation on the hematopoietic stem cell compartment [6-9]. In mice, the radiation sensitivity of hematopoietic progenitor cells has been measured based upon these cells' marrow repopulating ability post-radiation exposure, as well as the colony forming unit-spleen (CFU-S) assay [6,9]. A dose of 500 cGy has been shown to eliminate 99% of the competent hematopoietic stem cells based upon their ability to repopulate a lethally irradiated secondary recipient [6].

Conversely, it has been proposed that a small fraction of hematopoietic stem cells may be radio-resistant [9-11]. This hypothesis derives from observations in mice that a fraction of cells with CFU-S activity could survive doses of radiation up to 600 cGy [9]. Anecdotal observations of endogenous hematopoietic recovery in humans following exposure to nuclear fallout [12,13] have also suggested this possibility. Low dose radiation induces cellular apoptosis via activation of Fas-ligand mediated pathways [14,15], whereas higher dose radiation induces double-stranded DNA damage, which is most rapidly lethal in dividing cells [16]. Since the majority of primitive BM stem cells are quiescent in the steady state [17,18], the present invention considers it plausible that such cells might possess the capacity to repair radiation-induced DNA damage.

Ionizing radiation abrogates hematopoietic function via deleterious effects on both hematopoietic cells and the marrow stromal microenvironment [19,20]. Direct toxic effects of irradiation on stromal cell lines have been demonstrated [20]. Irradiated stromal cells release nitric oxide, which in theory could contribute to the demise of neighboring hematopoietic stem cells in vivo [21]. Similarly, the potential for endothelial cells to provide abilities critical to hematopoietic recovery post-radiation exposure has recently been demonstrated [22]. Various cytokines, including IL-1, TNF-alpha, flt-3 ligand, SCF, and TPO have radioprotective effects in mice when administered prior to or immediately at the time of radiation exposure [23-26].

Original studies in mice have demonstrated the radiosensitivity of hematopoietic stem and progenitor cells [6-9, 35]. The radiosensitivity (Do) of the most primitive assayable hematopoietic progenitor cells has been estimated to range from 0.71 to 1.38 Gy with 99% of measurable long-term repopulating cells being eliminated following exposure to 500-600 cGy [6,8]. Few studies have examined the capacity for in-vitro culture to rescue stem cells following high dose radiation injury [36,37]. Pulse exposure of human BM CD34⁺ cells to TNF-alpha within the first hour following 0.45-9 Gy x-irradiation was shown to improve CD34⁺ cell recovery, but the beneficial effect was maximal at lower radiation doses [36]. More recently, in-vitro culture of human CD34⁺ cells with TPO, SCF, Flt-3 ligand and IL-3 within 30 minutes of 2.5 Gy exposure prevented apoptosis in 15% of the irradiated cells [37]. In-vivo assays to assess the repopulating capacity of the recovered populations were not performed in these studies [36,37].

Several pre-clinical studies have demonstrated that the in-vivo administration of cytokines can promote early hematopoietic recovery following sublethal radiation [46-50]. Additional studies have indicated that the in-vivo administration of IL-1, TNF-alpha, flt-3 ligand, SCF, or TPO prior to or within 2 hr following radiation exposure can be radioprotective [24,25,36,51,52]. Recently, the administration of SCF, Flt-3 ligand, TPO, IL-3, and SDF-1 to B6D2F1 mice 2 hrs following 800 cGy resulted in 87.5% 30 day survival as compared to 8.3% survival in control animals [53]. Since the benefit of cytokine administration diminishes significantly if given more than 2 hours post-radiation [24,25,51], the practical application of cytokine treatments for mass casualty victims of radiation injury may be limited.

Of particular note is applicant's previous publication, Chute et al, Military Medicine 167(2 supp.): 74-77 (February 2002) [58] where hematopoietic stem cells were culturable in coculture and irradiated MNCs gave rise to CFU-GM, BFU-e and CFU-Mix. However, when the bone marrow MNC were attempted to be cultured in simple liquid culture (without co-culture monolayers) did not maintained a significant number of hematopoietic cells post irradiation and did not give rise to CFU-GM, BFU-e or CFU-Mix.

### SUMMARY OF THE INVENTION

The present invention seeks to rescue individuals exposed to radiation or other treatment which causes harmful myeloablation by removing exposed hematopoietic cells, typically from the bone marrow or peripheral blood, culturing these cells ex-vivo and reintroducing cultured cells into the same individual.

The present invention also encompasses a cell culture system and reagents for irradiated hematopoietic stem cells, which produces cells suitable for autologous engraftment.

### BRIEF DESCRIPTION OF THE DRAWINGS

FIG. 1 shows a Schema of experimental transplantation procedures.

FIG. 2 displays data for culturing and PMVEC co-culturing and recovery of 1050 cGy irradiated BM cells.

FIG. 3 displays data for recovery of 1050 cGy irradiated BM colony forming cells.

FIG. 4 displays Cell cycle status of normal BM MNC versus irradiated BM MNC versus irradiated/PMVEC-cultured BM.

FIG. 5 is a Kaplan-Meier survival curve following Transplantation of 1050 cGy irradiated/ex vivo-cultured cells and controls.

FIG. 6 is a Scatter plot of donor cell engraftment.

FIG. 7 is a representative lineage engraftment of irradiated/PMVEC-cultured cells in the peripheral blood at 8 weeks post-transplantation.

### DESCRIPTION OF THE PREFERRED EMBODIMENTS

The present specification presents certain theories of why hematopoietic stem cells are acting in the fashion observed. The nature of hematopoietic stem cells and engraftment of bone marrow is poorly understood and it is likely to be better explained in the future. While applicant does not wish to be bound by such theories, they serve a role in understanding the invention and comparison to the prior art.

High dose ionizing radiation can cause lethal myeloablation in exposed individuals. The present invention demonstrates that functional BM stem cells can be rescued ex-vivo following harvest from lethally irradiated animals. These results have important implications for the management of victims of ionizing radiation injury. Heretofore, victims of high dose radiation injury have typically succumbed to the sequelae of BM aplasia (infections, bleeding) despite optimized supportive care and, in some cases, allogeneic stem cell transplantation [5]. The present invention of early harvest after radiation exposure and ex-vivo culture of autologous BM cells from radiation victims can generate classes of repopulating cells capable of providing both early radioprotection and long-term hematopoietic reconstitution.

While the present invention is exemplified by using irradiated individuals, any individual exposed to a hematopoietic stem cell harming condition is a candidate for the same treatment. Classes of examples include exposure to radiation, chemicals, toxins, microbial infection, autoimmune disorder and neoplasms. In each of these situations, the individual presents with a bone marrow harmed condition. The condition may be simple (radiation/chemical/toxin exposure) or progressive (ongoing infection, cancer or other disorder such as aplastic anemia and other myelodysphasic syndromes).

The source of hematopoietic stem cells or other bone marrow cells harmed may be from peripheral blood (particularly after chemical mobilization), bone marrow aspirate, bone marrow biopsy or other location known or suspected of containing such cells. Cord blood is also a rich source of hematopoietic stem cells which may also be used in the rare situation where the fetus is exposed prenatally. The amounts needed to be recovered depend on the degree of myeloablation. When only part of the body is exposed to the harmful effect, hematopoietic stem cells are preferably taken from unaffected or lesser-affected areas.

Culture conditions are variable in duration and culture medium but should contain sufficient cytokines and combinations of cytokines to support growth of hematopoietic stem cells in vitro. Preferred cytokines include stem cell factors, interleukins, colony stimulating factors, Flt-3 ligand, erythropoietin, tumor necrosis factors, thrombopoietin and combinations of plural cytokines. The cytokines may be provided individually pure form or extracts, conditioned mediums and the like. Particularly preferred is PLURIGEN™, Large Scale Biology Corp., Vacaville, Calif. The cytokines may be from the same species as the individual being treated or from a different species.

Co-culturing cells may be stromal cells, endothelial cells, particularly vascular or microvascular endothelial cells. Particularly preferred are PMVEC and HUBEC cells. Extracts and conditioned medium from these cells may also be used.

The ex-vivo culturing may be sufficient to cause an increase in cell number, function, or differentiation. Alternatively, maintenance or even a dramatic decrease in the number of cells in culture may be acceptable provided that the ability to engraft the individual is maintained. While not wishing to be bound by theory, inactivation or death of hematopoietic stem cells in vivo may be due to the effects of radiation and the like on cells adjacent to or even peripheral to certain hematopoietic stem cells. Thus, simple removal of critical cells until the individual has cleared the harmful damage may be all that is required. Alternatively, extraction of exposed stem cells from the injured marrow microenvironment and short-term culture in an optimized ex-vivo environment could promote stem cell repair following exposure even without cell growth.

The present invention demonstrates for the first time that hematopoietic cells with short and long term repopulating capacity can be rescued via ex-vivo culture following harvest from lethally irradiated animals. 1050 cGy irradiation eliminated BM CFC and cells capable of in-vivo radioprotection in lethally irradiated secondary animals. In contrast, co-culture of 1050 cGy irradiated BM MNC with PMVEC+GM36SF supported the recovery of 69% of the input hematopoietic cells, 41% of the CFC, and, most importantly, cells with in-vivo radioprotective and long-term repopulating capacity. Liquid culture of 1050 cGy irradiated BM MNC with GM36SF alone supported significantly less recovery of input cells and CFC, but did sustain a population of cells capable of providing radioprotection and repopulation in recipient mice. These results demonstrate the therapeutic potential of ex-vivo culture to rescue functional hematopoietic cells following high dose radiation injury.

Comparison of the capacity of PMVEC co-culture and GM36SF alone to rescue irradiated BM stem cells revealed important similarities and differences. First, both PMVEC cultures and GM36SF-cultures rescued subsets of irradiated BM cells capable of providing radioprotection and multilineage repopulation in lethally irradiated recipients. Although previous publications [27-29] have shown the capacity of brain endothelial cells to support the expansion of BM cells with long-term repopulating capacity, previous studies have demonstrated that ex-vivo culture of normal BM stem cells with cytokines alone results in a loss of repopulating capacity [38-42]. The present invention shows that a population of primitive BM cells with limited radiosensitivity can be cultivated ex-vivo under conditions both previously suggested to support (PMVEC) and not previously suggested to support (GM36SF) stem cell recovery. PMVEC co-culture supplemented with GM36SF alone was significantly more potent (4-fold) than GM36SF alone toward recovering functional BM stem and progenitor cells post-high dose radiation. Whereas in a mouse system co-culture of BM MNC from 1-2 1050 cGy irradiated donor mice with PMVEC would have yielded adequate cells (≧2×10⁶ cells) to radioprotect irradiated recipient mice, marrow from 6-8 donor mice would be required for culture with GM36SF alone to yield adequate numbers of cells to provide the same in vivo activity. Extended culture duration and/or the addition of other cytokine combinations, such as PLURIGEN™ (Large Scale Biology Corp., Vacaville, Calif.) or TPO+SCF+Flt-3 ligand [43], and/or other cytokines or conditioned mediums, with and without PMVEC, HUBEC or other endothelial cells, can further optimize the recovery and expansion of irradiated repopulating cells and lineage-specific subsets [44,45].

The data in the present invention indicates that ex-vivo culture of marrow stem cells 6-8 hours following 1050cGy allows for the recovery of radioprotective and long-term repopulating cells. It is believed that longer durations post radiation exposure may be effective for ex-vivo culture. Marrow retrieved about 1-10 days following radiation exposure is also believed to provide similar benefit and animals transplanted with ex-vivo cultured cells these longer times post-radiation injury should achieve at least some survival advantage.

In another embodiment of the present invention is a method for culturing exposed hematopoietic stem cells in a cytokine containing culture medium without other supporting cells. This medium is capable of maintaining or repairing the engraftment potential of the exposed hematopoietic stem cells. The medium is preferably capable of proliferating or differentiating the cells in a manner that enhances their engraftment abilities.

The culture itself is a novel composition as well as pharmaceutically acceptable compositions containing the cultured exposed hematopoietic stem cells as the active ingredient in an acceptable vehicle. This composition is preferably used autologously but may be used for allogeneic stem cell transplantation as well with appropriately tissue-matched recipients.

### EXAMPLE 1

### Ex-Vivo Culture of Irradiated Bone Marrow Cells

PMVEC cultures and stroma-free liquid cultures were supplemented with GMCSF, IL-3, IL-6, SCF, Flt-3 ligand (GM36SF—liquid media) as previously described [17]. PMVEC were plated at 1×10⁵ cells/well in gelatin-coated 6-well plates (Costar, Cambridge, Mass.) containing 5 mL of M199 (Invitrogen, Carlsbad, Calif.), 10% FCS (Hyclone, Logan, Utah), 100 mcg/mL L-glutamine, 50 mcg/mL heparin, 30 mcg/mL endothelial cell growth supplement (Sigma, St. Louis, Mo.) and 100 mcg/mL penicillin/streptomycin. After 72 hr, the adherent PMVEC monolayers were washed with PBS and 5 mL of IMDM (Invitrogen) supplemented with 10% FCS, 1% pcn/strep, 2 ng/mL mu-GM-CSF, 5 ng/mL mu-IL-3, 5 ng/mL mu-IL-6, 120 ng/mL mu-SCF, and 50 ng/mL mu-Flt-3 ligand (R & D Systems, Minneapolis, Minn.) were added.

4×10⁵ BM MNC, which had been harvested from 1050 cGy (550 cGy and 500 cGy split by 4 hrs) irradiated and normal mice, were added to each well and incubated at 37° C. in humidified 5% CO₂-in-air atmosphere. After 7 days, an additional 3 mL of culture medium were added to each well. At day 10, the monolayers were washed to remove all non-adherent cells and the viable collected cells were counted using trypan blue exclusion dye. Irradiated BM MNC were also plated in liquid suspension cultures with liquid GM36SF as controls.

The data showing cell growth in both co-culture and with the cytokine-containing medium alone are shown as FIG. 2. FIG. 2 (A): Normal BM MNC (4×10⁵) were placed in co-culture with PMVEC+GM36SF or GM36SF alone×10 days. Hematopoietic cell counts (y axis) were measured at days 0, 4, 7, and 10 in each group. The mean cell counts at each time point are shown (n=6 experiments). FIG. 2 (B): BM MNC (4×10⁵) harvested from mice irradiated with 1050 cGy split dose were placed in culture with PMVEC+GM36SF or GM36SF alone×10 days. The mean cell counts at days 0, 4, 7, and 10 in each group are shown (n=6).

The total cell expansion of non-irradiated BM MNC during co-culture with PMVEC+GM36SF was not significantly different than that observed with GM36SF alone over 10 days (FIG. 2A). In contrast, when 1050cGy irradiated BM MNC were placed in liquid suspension cultures supplemented with GM36SF, an 89% decline in cell numbers by day 4 and only 18% of the starting population was recovered at day 10 (FIG. 2B; n=6) was observed. Conversely, 1050cGy irradiated BM MNC which were cultured with PMVEC+GM36SF declined by 62% by day 4, but subsequently an increase in cell numbers such that the viable cell count at day 10 recovered to 69% of the input number was observed. The difference in recovery of irradiated cells following PMVEC co-culture (Day 10: 2.8×10⁵±0.7 cells; Input: 4×10⁵ BM MNC) compared to GM36SF alone (Day 10: 7.0×10⁴±0.6 cells; Input: 4×10⁵ BM MNC) was highly significant (p=0.002; Wilcoxon rank sum test). Similarly, the percentage of non-viable cells in GM36SF-cultures at each time point was 40-50% as compared to <10% within PMVEC-cultures, indicating differences in the recovery process in these two groups following radiation exposure.

Stromal cell free cultures were also repeated using a cytokine supplement on only TPO, SCF and Flt-3 ligand at usual concentrations. Similar cell growth and engraftment data resulted with this alternative cytokine mixture.

### EXAMPLE 2

### Analysis of Irradiated Bone Marrow Cells

Colony forming assays were performed using a modification of the technique previously described [17]. Briefly, 5-50×10² BM MNC were seeded into 2 mL of IMDM, 1% methylcellulose, 30% FCS, 10 U/mL mu-erythropoietin, 2 ng/mL mu-GM-CSF, 10 ng/mL mu-IL-3, and 120 ng/ml mu-SCF. After 14 days, CFU-GM, BFU-E, and CFU-Mix colonies (>50 cells) were counted in each group. Triplicate assays were set up for each individual data point per experiment.

FIG. 3 displays the results of the assay for colony forming cells (CFC). Bone marrow MNC were placed in 14 day methylcellulose cultures to measure colony forming cell frequency. The bar graphs show the mean number of CFU-GM, BFU-E, CFU-Mix, and CFU-Total measured at day 14 from each of 3 groups (n=5): At left, the CFC content within Normal BM MNC is shown; the middle bar shows the CFC content of 1050 cGy Irradiated/PMVEC-cultured cells; at right, the CFC content within 1050 cGy irradiated cells cultured with GM36SF alone is shown. Of note, no CFC were recovered within 1050 cGy irradiated BM MNC which were not subsequently cultured (data not shown). *The difference in CFC content between 1050 cGy irradiated/PMVEC-cultured cells vs. 1050 cGy irradiated/GM36SF cultured cells was highly significant.

The CFC frequency within 1050cGy irradiated BM MNC, 1050cGy irradiated/GM36SF-cultured cells and 1050cGy irradiated/PMVEC-cultured cells, and compared these with the CFC content of normal BM MNC. Bone marrow MNC obtained from 1050 cGy irradiated mice showed no measurable CFU-GM, BFU-E, or CFU-Mix (n=5). As shown in FIG. 3, culture with GM36SF alone supported the recovery of <1% of the CFU-GM, BFU-E, and CFU-Mix contained in normal BM (n=5). Conversely, day 10 PMVEC cultures contained 44% as many CFU-GM as compared to normal BM MNC, 32% of the BFU-E, 32% of the CFU-Mix, and 41% of the CFU-total (n=5)(FIG. 3). The recovery of CFC in PMVEC cultures was significantly greater than the CFC recovery observed in cultures with GM36SF alone (p=0.0079; Wilcoxon rank sum test).

Cell cycle analysis and phenotype of 1050cGy irradiated and ex vivo cultured BM cells Normal BM MNC, 1050cGy irradiated BM MNC, and 1050cGy irradiated/PMVEC-cultured cells were stained with propidium iodide (PI, Molecular Probes, Eugene, Oreg.) for DNA content analysis as previously described [33]. Briefly, BM MNC were fixed in 70% ethanol for >2 hours at 4° C., then washed with PBS and then resuspended in 0.1% Triton X-100 (Sigma), 0.2 mg/ml RNase A (Qiagen), and 20 μg/ml propidium iodide. Cells were analyzed using a BD FACScalibur. Histogram analysis was conducted using ModFIT LT software (Verity Software House, Topsham, Me.). At least 10,000 events were collected for analysis of each sample.

Phenotypic analysis of BM MNC pre- and post-PMVEC culture was performed using anti-murine CD3 PE, anti-B220 PE, and anti-Mac-1 PE and compared to isotype control staining.

FIG. 4 depicts the cell cycle status of normal BM MNC versus irradiated BM MNC versus irradiated/PMVEC-cultured BM. Normal BM MNC, 1050 cGy irradiated BM MNC, and 1050 cGy/PMVEC-cultured BM cells were stained with propidium iodide to measure cellular DNA content and cell cycle status. Day 0 normal (non-irradiated) BM MNC (A) and 1050 cGy irradiated BM MNC (B) resided primarily (>80%) in G₀/G₁ phase as evidenced by the predominant population in the left peak. A smaller percentage of cells is evident in G₂+M phase (right peak) and S phase (intermediate population). Following 10 days of PMVEC co-culture (C), a significant increase in cells in S phase and G₂+M phase was observed as shown. As a control, normal BM MNC cultured with PMVEC are also shown (D).

As represented in FIGS. 4A & B, a mean of 84% of normal BM MNC and 82% of 1050cGy irradiated BM MNC were found to reside in G₀/G₁ phase at day 0. Interestingly, when 1050cGy irradiated BM MNC were cultured with PMVEC, the G₀/G₁ fraction decreased to 66% of the total while 16% and 18% entered S and G₂+M phase, respectively (FIG. 4C). Normal BM MNC cultured with PMVEC showed slightly less proliferation, with 72% remaining in G₀/G₁, and 28% residing in S and G₂+M phase (FIG. 4D). These data suggests that PMVEC co-culture did not induce quiescence in irradiated stem/progenitor cells as a mechanism of recovery, but appeared to induce proliferation of BM cells during the repair period.

Phenotype analysis of BM MNC pre- and post-exposure to 1050 cGy revealed an immediate lymphotoxic effect of ionizing radiation. Normal BM MNC contained 30.5%±0.7 Mac-1⁺ cells, 47.4%±2.5 B-220+cells, and 2.0%±0.2 CD3⁺ cells (n=6). Six hours post-1050cGy radiation, the proportion of cells changed to 52.7%±0.9 Mac-1⁺, 31.6%±0.9 B-220⁺, and 4.9%±0.5 CD3⁺ cells, indicating a relative loss of B cells. Following PMVEC co-culture, the 1050cGy irradiated population became significantly enriched for cells lacking lineage commitment: 18.3%±0.2 Mac-1⁺, 2.9%±0.3 B220⁺, and 0.9%±0.1 CD3⁺. These data confirmed that the combination of 1050cGy irradiation followed by PMVEC co-culture selectively enriched for phenotypically immature hematopoietic cells. For comparison, 93% of normal BM MNC cultured with PMVEC×10 days expressed lineage-specific markers (mean 91% Mac-1⁺)(data not shown).

### EXAMPLE 3

### Transplantation of Irradiated BM Cells into Irradiated Recipient Mice

Syngeneic 12 week old B6.5JL (CD45.1) males and C57BL6 (CD45.2) females were used as donor and recipient mice, respectively (Jackson Laboratories, Sacramento, Calif.) [34]. Donor mice were irradiated with a split dose of 1050cGy delivered by a linear accelerator at 100 cGy/minute. Two hours post-radiation, donor animals were sacrificed and their BM was collected by flushing their femurs with 4° C. PBS plus 10% FCS. The cells were washed×2 and the MNC were collected using Ficoll-Hypaque separation (Amersham Biosciences, Piscataway, N.J.). Six to 8 hours post-radiation exposure, the BM MNC were then either utilized for tail vein injections into recipient mice or placed in expansion cultures. 1050cGy irradiated CD45.2 recipient mice were divided into 5 treatment groups as shown in FIG. 1: 1) Recipient mice transplanted with 1050cGy irradiated donor BM MNC, 2) mice transplanted with 1050cGy irradiated/PMVEC-cultured cells, 3) mice transplanted with 1050cGy irradiated/GM36SF-cultured cells, 4) mice transplanted with non-irradiated donor BM MNC, and 5) mice transplanted with PMVEC alone. As a negative control, a group of 1050cGy irradiated recipient mice were also followed without a donor cell infusion. All animals were given acidified water supplemented with Sulfa/Trimethoprim suspension (1%, Alpharma, Baltimore, Md.) commencing at day 0. A 1050cGy split dose was chosen because it was previously found this to be the LD_{100/30} for 12-week-old C57B16 mice.

FIG. 1 depicts a schema of experimental transplantation procedures. As shown at the top, C57B16 donor mice (CD 45.1) were irradiated with 1050 cGy (split dose) and their bone marrow (BM) was subsequently harvested. Purified BM mononuclear cells (MNC) were obtained via Ficoll-Hypaque centrifugation. A group of CD 45.2 recipient mice were irradiated with 1050 cGy and then transplanted via tail vein injection with 2×10⁶ irradiated BM MNC per mouse. A second group of donor CD 45.1 mice were irradiated with 1050 cGy, had BM MNC collected identically and these cells were placed in co-culture with PMVEC+GM36SF×10 days. At day 10, the non-adherent hematopoietic cells were collected from these cultures and injected via tail vein injection into irradiated CD 45.2 mice at doses of 2-4×10⁶ cells per mouse (middle). As a positive control (bottom), CD 45.2 mice were irradiated with 1050 cGy and then transplanted with 2×10⁶ normal CD 45.1 donor BM MNC. An additional group of CD 45.2 mice was irradiated with 1050 cGy and followed for survival without donor cell infusion. All animals were followed for 8 weeks post-transplantation for survival and donor engraftment.

As shown above in FIG. 2, PMVEC co-culture supported an average 4-fold greater recovery of viable BM cells following 1050cGy irradiation as compared to GM36SF alone. In order to perform transplantation studies with a minimum dose of 2×10⁶ cells per recipient mouse, 5 independent experiments were performed in which 1050cGy irradiated donor mice were sacrificed, BM MNC collected, and the cells placed in PMVEC co-culture prior to injection. On average, a single 1050cGy irradiated mouse yielded 2-3×10⁶ BM MNC, BM collected from 5-10 donor mice in each experiment was pooled to yield adequate cell numbers for subsequent transplantation into recipient mice. For example, in one experiment, 10 donors were irradiated, 2×10⁷ BM MNC were collected and plated on PMVEC. At day 10, 1.2×107 viable cells were collected and 6 mice were transplanted with 2×10⁶ cells each. In contrast, in order to collect an equal number of viable hematopoietic cells following GM36SF culture of 1050cGy irradiated BM MNC, 40 donor mice were sacrificed (8×10⁷ input BM MNC) over 2 separate experiments which subsequently yielded a total of 1.3×10⁷ viable cells at day 10. Therefore, PMVEC culture was 3-4 times more potent than GM36SF alone toward recovery of transplantable grafts following 1050 cGy.

FIG. 5 shows the survival of 1050 cGy irradiated recipient mice after receiving transplants from 1050 cGy irradiated/ex vivo-cultured cells. Kaplan-Meier curves are shown demonstrating the survival of 7 groups of 1050 cGy irradiated CD45.2 recipient mice: 1) Mice (n=10) followed without cell transplant (filled circles), 2) mice (n=9) transplanted with 1050 cGy irradiated donor BM MNC (2×10⁶)(filled squares), 3) mice (n=10) transplanted with 1050 cGy irradiated/PMVEC-cultured cells (2×10⁶ cells; triangles), 4) mice (n=6) transplanted with 1050 cGy irradiated/PMVEC-cultured cells (4×10⁶ cells per mouse; stars), 5) mice (n=10) transplanted with 2×10⁶ normal BM MNC (diamonds), 6) mice (n=8) transplanted with 1050 cGy irradiated/GM36SF-cultured cells (2×10 6; open squares), and 7) mice (n=10) transplanted with PMVEC alone (2×10⁶; open circles). All mice in each group have been followed for a minimum of 8 weeks following irradiation with 1050 cGy (X axis). The Y-axis shows the percent of animals surviving at each time point.

One hundred percent of recipient mice transplanted with 2×10⁶ normal donor BM MNC remained well through week 8. Conversely, all recipient mice followed without cell infusion died by day 18. Similarly, all recipient mice, which were transfused with 2×10⁶ 1050cGy, irradiated donor BM MNC died by day 27. In contrast, when recipient mice were transplanted with 2×10⁶ 1050cGy irradiated/PMVEC-cultured cells, 60% survived through week 8. When an additional group of recipient mice was transplanted with 4×10⁶ irradiated/PMVEC-cultured cells, all remained well through week 8 (n=5 experiments). The percent survival of recipient mice transplanted with 2 or 4×10⁶ irradiated/PMVEC-cultured cells was significantly greater than the survival of mice which received no transplant (p=0.0009 and p=0.0008, respectively; exact log rank test). Similarly, the survival of mice transplanted with either 2 or 4×10⁶ irradiated/PMVEC-cultured cells was significantly greater than the survival of recipient mice which received 2×10⁶ irradiated BM MNC (p=0.02 and p=0.001, respectively). The survival of mice transplanted with 2×10⁶ irradiated/PMVEC-cultured cells was not significantly different than the survival of animals rescued with 2×10⁶ normal BM MNC (p=0.26).

For comparison, a group of mice with 2×10⁶ 1050cGy irradiated/GM36SF-cultured cells (n=3 experiments) was transplanted. Fifty percent of these mice survived through week 8, indicating that a percentage of radioprotective cells were also maintained by GM36SF alone in the absence of PMVEC. 10 mice with 2×10⁶ PMVEC following 10-day culture with GM36SF were transplanted. Twenty percent of these mice survived through week 8, indicating that PMVEC alone were capable of imparting radioprotection in vivo across xenotransplantation barriers. The 8-week survival of mice transplanted with 1050cGy irradiated/PMVEC-cultured cells was not significantly different than the survival of mice transplanted with 1050cGy irradiated/GM36SF-cultured cells (p=0.6), but was significantly better than mice transplanted with PMVEC alone (p=0.05).

FIG. 6 shows a scatter plot of donor (CD 45.1⁺) cell engraftment in CD 45.2+mice. The engraftment of donor CD 45.1 cells in the peripheral blood 8 weeks following transplantation into 1050 cGy irradiated CD 45.2 recipient mice is shown. The far left column shows the engraftment of normal BM MNC transplanted at 2×10⁶ cells per mouse. The engraftment percentages of 1050 cGy irradiated donor cells, 1050 cGy irradiated/PMVEC-cultured cells, 1050 cGy irradiated/GM36SF-cultured cells, and PMVEC alone are shown to the right. Animals that survived >8 weeks are represented by open circles. Animals that died prior to 30 days are represented by filled circles.

This measured the capacity for PMVEC-cultured and GM36SF-cultured cells to provide donor repopulation in recipient mice. As shown in FIG. 6, non-irradiated BM MNC repopulated 1050cGy irradiated recipient mice at high levels (mean 72.7% CD45.1⁺ cells), whereas 1050cGy irradiated BM MNC failed to repopulate recipient mice. Conversely, 33% of surviving mice transplanted with 2×10⁶ 1050cGy irradiated/PMVEC-cultured cells showed donor CD45.1⁺ cells (>1%) in the peripheral blood at week 8. In mice transplanted with 4×10⁶ 1050cGy irradiated/PMVEC cultured cells, 67% showed donor CD45.1⁺ cell engraftment with 2-fold higher levels of circulating CD45.1⁺ cells than in the lower dose group. Mice transplanted with 1050cGy irradiated/GM36SF-cultured cells showed CD45.1⁺ cell engraftment in 2 of 4 mice (50%) that survived through week 8, at levels comparable to mice transplanted with PMVEC-cultured cells at the same dose.

Engraftment of CD45.1 cells in CD45.2 mice was measured in the peripheral blood (PB) via flow cytometry at week 8 post-transplantation. PB samples were treated with RBC Lysis buffer (Sigma)×30 minutes, then washed and stained with anti-CD45.1 FITC and anti-CD45.2 FITC (BD) and compared with isotype control staining. Lineage engraftment was also measured at week 8. PB samples were stained with anti-B220 PE, anti-CD3 PE, and anti-Mac-1 PE (BD) to assess for B-lymphoid, T-lymphoid, and myeloid reconstitution.

FIG. 7 shows the representative lineage engraftment of irradiated/PMVEC-cultured CD 45.1⁺ cells in the peripheral blood of CD 45.2+mice at 8 weeks post-transplantation. (A) As a control, the dot plot of peripheral blood cells from a CD 45.2⁺ mouse, which received no transplant, is shown. Isotype control staining is shown at left and staining with CD 45.1 FITC is shown at right. (B) Dot plot of PB cells from a representative CD 45.2+mouse transplanted with 1050 cGy irradiated/PMVEC-cultured CD 45.1⁺ cells. Isotype staining is shown at left and CD 45 FITC staining is shown at right. (C) Expression of Mac-I PE, CD 3 PE, and B220 PE (y axis) on engrafted donor CD 45.1⁺ cells in a mouse transplanted with 1050 cGy irradiated/PMVEC-cultured cells. Isotype staining is shown in upper left.

The donor CD45.1⁺ cell engraftment in a representative mouse transplanted with 1050cGy irradiated/PMVEC cultured cells is shown in FIG. 7. Of note, myeloid, T-lymphoid, and B-lymphoid donor engraftment was observed in mice transplanted with 1050cGy irradiated/PMVEC cultured cells, indicating that primitive hematopoetic cells with differentiative capacity were rescued following the high dose radiation exposure. However, as shown in FIG. 7C, the percentages of engrafted donor cells expressing Mac-I and B220 were <10% in these mice, whereas the percentage of CD3⁺ cells was significantly higher (mean 75.4%). This lineage distribution paralleled the high percentage of CD3⁺ cells evident within the endogenous recovering population in the irradiated mice. Table 1 summarizes the CD45.1⁺ cell engraftment and lineage distribution in all mice transplanted with 1050cGy irradiated/PMVEC-cultured cells, 1050cGy irradiated/GM36SF-cultured cells, and normal BM MNC. Of note, mice transplanted with 1050cGy irradiated/PMVEC-cultured cells have subsequently been followed for >6 months from the time of transplantation and all mice which showed donor engraftment at 8 weeks have continued to demonstrate multilineage donor repopulation.

TABLE 1
Engraftment of 1050cGy Irradiated/Ex-vivo cultured
BM vs. Normal BM MNC in recipient mice

| | | | Percent of CD 45.1⁺ donor | Percent of engrafted | | |
|---|---|---|---|---|---|---|
| | | | cells in peripheral blood of | CD 45.1⁺ cells expressing: | | |
| Group | Cell Dose | Survival | recipient CD 45.2⁺ mice | B220 | Mac-1 | CD3 |
| Irradiated | 2 × 10⁶ | 4/8 | 3.1% ± 5.3 | 43.0% ± 9.9 | 25.8% ± 10.6 | 32.5% ± 24.7 |
| + | | | (range 0-11.0%) | | | |
| GM36SF | | | | | | |
| Irradiated | 2 × 10⁶ | 6/10 | 5.0% ± 10.7 | 5.9% ± 3.3 | 3.7% ± 2.1 | 75.4% ± 14.3 |
| + | | | (range: 0-26.8%) | | | |
| PMVEC | | | | | | |
| culture | 4 × 10⁶ | 6/6 | 10.8% ± 10.9 | | | |
| | | | (range 0.1-30.3%) | | | |
| Normal | 2 × 10⁶ | 10/10 | 72.7% ± 12.6 | 51.2% ± 8.5 | 8.7% ± 2.6 | 10.4% ± 3.3 |
| BM MNC | | | (range: 49-85.2%) | | | |

The CD 45.1⁺ donor cell engraftment reflects the mean percent CD 45.1⁺ cells detectable in the peripheral blood of all mice, which survived at least 8 weeks post-irradiation. This includes recipient mice, which survived but had <1% engraftment of donor CD45.1⁺ cells. Measurements of B220, Mac-1, and CD 3 lineage engraftment were made at 8 weeks post-transplantation in mice which demonstrated >1% CD 45.1⁺ cell engraftment.

It will be understood that various modifications may be made to the embodiments disclosed herein. Therefore, the above description should not be construed as limiting, but merely as exemplifications of preferred embodiments. Those skilled in the art will envision other modifications within the scope and spirit of the claims appended hereto.

All patents and references cited herein are explicitly incorporated by reference in their entirety.

### REFERENCES

1. Mettler F, Voelz G. Major radiation exposure—what to expect and how to respond. N Engl J Med 2002;346:1554-61.
2. Dainiak N. Radiation response: changing concepts and emerging paradigms. Exp Hematol. 2003;31:435-6.
3. Dainiak N. Hematologic consequences of exposure to ionizing radiation. Exp Hematol. 2002;30:513-28.
4. NCRP Report No. 138. Management of terrorist events involving radioactive material. 2001; p. 37
5. Hirama T, Tanosaki S, Kandatsu S, et al. Initial medical management of patients severely irradiated in the Tokai-mura criticality accident. Br J Radiol. 2003;76:246-53.
6. Meijne E, Ria J, van der Winden-van Groenewegen R, Ploemacher R, Vos 0, David J, Huiskamp R. The effects of x-irradiation on the hematopoietic stem cell compartment in the mouse. Exp Hematol. 1991;19:617-23.
7. McCarthy K. Population size and radiosensitivity of murine hematopoietic endogenous long term repopulating cells. Blood. 1997;89:834-41.
8. Down J, Boudewijn A, van Os R, Thames H, Ploemacher R. Variations in radiation sensitivity and repair among different hematopoietic stem cell subsets following fractionated irradiation. Blood. 1995;86:122-7.
9. Inoue T, Hirabayashi Y, Mitsui H, et al. Survival of spleen colony-forming units (CFU-S) of irradiated bone marrow cells in mice: evidence for the existence of a radioresistant subfraction. Exp Hematol. 1995;23:1296-1300.
10. van Bekkum D. Radiation sensitivity of the hematopoietic stem cell. Rad Res. 1991;128:S4-8.
11. Wagemaker G. Heterogeneity of radiation sensitivity of hematopoietic stem cell subsets. Stem Cells. 1995;13 Suppl:257-60.
12. Baranov A, Gale R, Guskova A, et al. Bone marrow transplantation after the Chernobyl nuclear accident. N Engl J. Med. 1989;321:254-5.

13. Dainiak N, Sorba S. Early identification of radiation accident victims for therapy of bone marrow failure. Stem Cells. 1997;15 Suppl 2:275-85.
14. Albanese J, Dainiak N. Ionizing radiation alters Fas ligand at the cell surface and on exfoliated plasma membrane-derived vesicles: implications for apoptosis and intercellular signaling. Rad Res. 2000; 153:49-61.
15. Belka C, Marini P, Budach W, et al. Radiation induced apoptosis in human lymphocytes and lymphoma cells critically relies on the up-regulation of CD9/Fas/APO-1 Ligand. Rad Res. 1998;149:588-95.
16. Radford I, Murphy T. Radiation response of mouse lymphoid and myeloid cell lines. Part III. Different signals can lead to apoptosis and may influence sensitivity to killing by DNA double-strand breakage. Int J Radiat Biol. 1994;65:229-39.
17. Chute J, Saini A, Kampen R, Wells M, Davis T. A comparative study of the cell cycle status and primitive cell adhesion molecule profile of human CD34⁺ cells cultured in stroma-free versus porcine microvascular endothelial cell cultures. Exp Hematol. 1999;27:370-7.
18. Petzer A, Zandstra P, Piret J, Eaves C. Differential cytokine effects on primitive (CD34⁺ CD38-) human hematopoietic cells: novel responses to flt-3 ligand and thrombopoietin. J Exp Med. 1996;183:2551-8.
19. Galotto M, Berisso G, Delfino L, et al. Stromal damage as a consequence of high dose chemo/radiotherapy in bone marrow transplant recipients. Exp Hematol. 1999;27: 1460-6.
20. Greenberger J, Anderson J, Berry L, et al. Effects of irradiation of CBA/CA mice on hematopoietic stem cells and stromal cells in long term bone marrow cultures. Leukemia. 1996;10:514-27.
21. Gorbunov N, Pogue-Geile K, Epperly M, et al. Activation of the nitric oxide synthase 2 pathway in the response of bone marrow stromal cells to high doses of ionizing radiation. Radiat Res. 2000;154:73-86.
22. Montfort M, Olivares C, Mulcahy J, Fleming W. Adult blood vessels restore host hematopoiesis following lethal irradiation. Exp Hematol. 2002;30:950-6.
23. Neta R, Oppenheim J, Douches S. Interdependence of the radioprotective effects of human recombinant interleukin 1 alpha, tumor necrosis factor alpha, granulocyte colony stimulating factor, and murine recombinant granulocyte macrophage colony-stimulating factor. J. Immunol. 1988;140:108-11.
24. Hudak S, Leach M, Xu Y, Menon S, Rennick D. Radioprotective effects of flk2/flt3 ligand. Exp Hematol. 1998;26:515-22.
25. Zsebo K, Smith K, Hartley C, et al. Radioprotection of mice by recombinant stem cell factor. Proc Natl Acad Sci USA. 1992;89:9464-8.
26. Pestina T, Cleveland J, Yang C, Zambetti G, Jackson C. Mpl ligand prevents lethal myelosuppression by inhibiting p53-dependent apoptosis. Blood. 2001;98:2084-90.
27. Chute J, Saini A, Chute D, et al. Ex vivo culture with human brain endothelial cells increases the SCID-repopulating capacity of adult human bone marrow. Blood. 2002;100:4433-9.
28. Brandt J, Galy A, Luens K, et al. Bone marrow repopulation by human marrow stem cells following long term expansion culture on a porcine endothelial cell line. Exp Hematol. 1998;26:950-61.
29. Brandt J, Bartholemew A, Fortman J, et al. Ex vivo expansion of autologous bone marrow CD34⁺ cells with porcine microvascular endothelial cells results in a graft capable of rescuing lethally irradiated baboons. Blood 1999;94: 106-13.
30. Shalaby F, Rossant J, Yamaguchi T, et al. Failure of blood island formation and vasculogenesis in flk-1 deficient mice. Nature. 1995;376:62-6.
31. Hamaguchi I, Huang X, Takakura N, et al. In vitro hematopoietic and endothelial development from cells expressing TEK receptor in murine aorta gonad mesonephros regioni. Blood. 1999;93:1549-56.
32. Choi K, Kennedy M, Kazarov A, et al. A common precursor for hematopoietic and endothelial cells. Development. 1998;125:725-32.
33. Mahmud N, Devine S, Weller K, et al. The relative quiescence of hematopoietic stem cells in nonhuman primates. Blood. 2001;97:3061-3068.
34. Zhao Y, Lin Y, Zhan Y, et al. Murine hematopoietic stem cell characterization and its regulation in BM transplantation. Blood. 2000;96:3016-22.
35. Ploemacher R, van Os R, van Beurden C, et al. Murine hemopoietic stem cells' long term engraftment and marrow repopulating ability are more resistant to gamma-radiation than are spleen colony forming cells. Int J Radiat Biol. 1992;61:489-99.
36. Karkanitsa L, Komarovskaya M, Krivenko S. Abrogation of radiation injury to human hematopoietic stem cells with tumor necrosis factor-alpha. Stem Cells. 1997;15 Suppl2:95-102.
37. Drouet M, Mathieu J, Grenier N, et al. The reduction of in vitro radiation-induced fas-related apoptosis in CD34⁺ progenitor cells by SCF, Flt-3 ligand, TPO, and IL-3 in combination resulted in CD34⁺ proliferation and differentiation. Stem Cells. 1999; 17:273-85.
38. Peters S, Kittler E, Ramshaw H, Quesenberry P. Ex vivo expansion of murine marrow cells with interleukin-3 (IL-3), IL-6, IL-11, and stem cell factor leads to impaired engraftment in irradiated hosts. Blood. 1995;87:30-7.
39. Szilvassy S, Bass M, Van Zant G, Grimes B. Organ selective homing define engraftment kinetics of murine hematopoietic stem cells and is compromised by ex-vivo expansion. Blood. 1999;93:1557-66.
40. Traycoff C, Cornetta K, Yoder M, Davidson A, Srour E. Ex vivo expansion of murine hematopoietic progenitor cells generates classes of expanded cells possessing different levels of bone marrow repopulating potential. Exp Hematol. 1996;24:299-306.
41. Varas F, Bernard A, Bueren J. Restrictions in the stem cell function of murine bone marrow grafts after ex vivo expansion of short term repopulating progenitors. Exp Hematol. 1998;26: 100-9.
42. Traycoff C, Orazi A, Ladd A, et al. Proliferation induced decline of primitive hematopoietic progenitor cell activity is coupled with an increase in apoptosis of ex vivo expanded CD34⁺ cells. Exp Hematol. 1998;26:53-62.
43. Tanavde V, Malehorn M, Lumkul R, et al. Human stem-progenitor cells from neonatal cord blood have greater hematopoietic expansion capacity than those from mobilized adult blood. Exp Hematol. 2002;30:816-23.
44. Louagie H, Van Eijkeren M, Philippe J, Thierens H, de Ridder L. Changes in peripheral blood lymphocyte subsets in patients undergoing radiotherapy. Int J Radiat Biol. 1999;75:767-71.
45. Prosser J. Survival of human T and B lymphocytes after X-irradiation. Int J Radiat Biol Relat Stud Phys Chem Med. 1976;30:459-65.
46. Morstyn G, Foote M, Perkins D, et al. The clinical utility of granulocyte colony-stimulating factor: early achievements and future promise. Stem Cells. 1994;12(suppl.1):213-27.

47. Monroy R, Skelly R, Taylor P, et al. Recovery from severe hematopoietic suppression using recombinant human granulocyte-macrophage colony-stimulating factor. Exp Hematol. 1988;16:344-8.
48. Ganser A, Lindemann A, Seipelt G, et al. Effects of recombinant human interleukin-3 in patients with normal hematopoiesis and in patients with bone marrow failure. Blood. 1990;76:666-76.
49. Patchen M, MacVittie T, Williams J, et al. Administration of interleukin-6 stimulates multilineage hematopoiesis and accelerates recovery from radiation-induced hematopoietic depression. Blood; 1991;77:472-80.
50. Patchen M, Fischer R, Schmauder-Chock E, Williams D. Mast cell growth factor enhances multilineage hematopoietic recovery in vivo following radiation-induced aplasia. Exp Hematol. 1994;22:31-9.
51. Mouthon M, Van der Meeren A, Gaugler M, et al. Thrombopoietin promotes hematopoietic recovery and survival after high-dose whole body irradiation. Int J Radiat Oncol Biol Phys. 1999;43:867-75.
52. Neta R, Oppenheim J. Cyokines in therapy of radiation injury. Blood. 1988;72:1093-5.
53. Herodin F, Bourin P, Mayol J, Lataillade J, Drouet M. Short-term injection of antiapoptotic cytokine combinations soon after lethal γ-irradiation promotes survival. Blood. 2003;101:2609-16.
54. Na Nakom T, Traver D, Weissman I, Akashi K. Myeloerythroid-restricted progenitors are sufficient to confer radioprotection and provide the majority of day 8 CFU-S. J Clin Invest. 2002;109:1579-85.
55. Wang M, Consoli U, Lane C, et al. Rescue from apoptosis in early (CD34-selected) versus late (non-CD34-selected) human hematopoietic cells by very late antigen 4 (VLA-4) and vascular cell adhesion molecule (VCAM) 1-dependent adhesion to bone marrow stromal cells. Cell Growth Differ. 1998;9:105-12.
56. Mugge A, Elwell J, Peterson T, Harrison D. Release of intact endothelium-derived relaxing factor depends on endothelial superoxide dismutase activity. Am J. Physiol. 1991;260:C219-25.
57. Epperly M, Bemarding M, Gretton J, et al. Overexpression of the transgene for manganese superoxide dismutase (MnSOD) in 32D cl 3 cells prevents apoptosis induction by TNF-α, IL-3 withdrawal, and ionizing radiation. Exp Hematol. 2003;31:465-74.
58. Chute JP, Clark W, Saini A, Wells M, Harlan D. Rescue of hematopoietic stem cells following high-dose radiation injury using ex vivo culture on endothelial monolayers. Mil Med 2002; 167(2 supp.): 74-7.

What is claimed is:

1. A method for autologous rescue of an individual exposed to a harmful hematopoietic stem cells condition comprising;
removing hematopoietic stem cells from said individual,
culturing the hematopoietic stem cells in a stromal-cell free medium, and
reintroducing cultured hematopoietic stem cells into said individual.

2. The method of claim 1 where the individual was exposed to radiation.

3. The method of claim 1 wherein the hematopoietic stem cells are removed within 10 days of said exposure.

4. The method of claim 3 wherein the hematopoietic stem cells are removed within one day of said exposure.

5. The method of claim 1 wherein the condition to which the individual was exposed is lethal if untreated.

6. The method of claim 1 wherein the hematopoietic stem cells are cultured for at least about 10 days.

7. The method of claim 1 wherein the hematopoietic stem cells are cultured in a medium containing thrombopoietin, stem cell factor and flt-3 ligand.

8. The method of claim 1 wherein the hematopoietic stem cells are cultured in a medium containing granulocyte-monocyte colony stimulating factor, interleukin-3, interleukin 6, stem cell factor and flt-3 ligand.

9. The method of claim 1 wherein the hematopoietic stem cells remain engrafted for at least 6 months.

10. A composition comprising viable hematopoietic stem cells previously exposed to a harmful hematopoietic stem cell condition, a cell culture medium, stem cell factor and flt-3 ligand.

11. The composition according to claim 10 further comprising thrombopoietin.

12. The composition according to claim 10 further comprising granulocyte-monocyte colony stimulating factor, interleukin-3 and interleukin 6.

13. The composition according to claim 10 capable of engrafting a lethally irradiated animal.

14. A method for culturing hematopoietic stem cells previously exposed to a harmful hematopoietic stem cell condition comprising;
contacting said cells in a cell culture medium containing cytokines, and
incubating said cells in said medium,
wherein the hematopoietic stem cells remain viable.

15. The method of claim 14 wherein the cytokines comprise stem cell factor.

16. The method of claim 15 wherein the cytokines further comprise thrombopoietin and flt-3 ligand.

17. The method of claim 15 wherein the cytokines further comprise granulocyte-monocyte colony stimulating factor, interleukin-3, interleukin 6 and flt-3 ligand.

18. The method of claim 14 wherein the hematopoietic stem cells are capable of engrafting a lethally irradiated animal.

19. A composition according to claim 10 that is a pharmaceutical composition.

20. A composition according to claim 13 that is a pharmaceutical composition.
